(19) United States
(12) Patent Application Publication
(10) Pub. No.: US 2024/0424274 A1
(43) Pub. Date: Dec. 26, 2024

(54) TRANSFER INSTRUMENT

(71) Applicant: TERUMO KABUSHIKI KAISHA, Tokyo (JP)

(72) Inventor: Koichi HAYAKAWA, Kanagawa (JP)

(73) Assignee: TERUMO KABUSHIKI KAISHA, Tokyo (JP)

(21) Appl. No.: 18/741,015

(22) Filed: Jun. 12, 2024

(30) Foreign Application Priority Data
Jun. 20, 2023 (JP) 2023-100694

(51) Int. Cl. A61M 37/00

(52) U.S. Cl. CPC A61M 37/0069; A61M 2202/0007; A61M 2202/06; A61M 2210/125

(57) ABSTRACT

In a transfer instrument, a medical sheet is supported by a first support surface of a first support portion and a second support surface of a second support portion. The second support portion has a shape tapered in a distal end direction of the transfer instrument.

### CROSS-REFERENCES TO RELATED APPLICATIONS

This application is based on and claims priority to Japanese Patent Application No. 2023-100694 filed on Jun. 20, 2023, the entire content of which is incorporated herein by reference.

### TECHNOLOGICAL FIELD

The present invention generally relates to a transfer instrument for transferring a medical sheet to a treatment site of a living body.

### BACKGROUND DISCUSSION

Japanese Patent Application Publication No. 2009-000511 (JP 2009-000511 A) discloses a transfer instrument for transferring a medical sheet (cell sheet) for use in, for example, organ transplantation to a treatment site of a living body. The transfer instrument includes an outer cylinder, a slide member slidably supported in the outer cylinder, and a seat support member provided at a distal end of the slide member.

### SUMMARY

It is desired to transfer a medical sheet to a treatment site more efficiently.

(1) A transfer instrument that is used to transfer a medical sheet to a treatment site of a living body includes: a first carrier member including a first shaft and a first support portion, the first support portion being provided at a distal end of the first shaft and having a first support surface; and a second carrier member including a second shaft and a second support portion, the second shaft extending along the first shaft and being movable along the first shaft, the second support portion being provided at a distal end of the second shaft and having a second support surface smaller than the first support surface, in which the first support surface and the second support surface are configured to support the medical sheet, and the second support portion has a shape tapered in a distal end direction.

With this configuration, it is possible to efficiently transfer, by moving the second shaft along the first shaft with the medical sheet supported by the first support surface and the second support surface, the medical sheet to the treatment site.

Further, with this configuration, since the second support portion has a shape tapered in the distal end direction, a contact area of the second support surface with the medical sheet becomes smaller toward the distal end, and frictional resistance between the medical sheet and the second support surface decreases accordingly. It is therefore possible to prevent, when the second shaft is moved in the proximal end direction along the first shaft after the medical sheet is transferred to the treatment site, the medical sheet from returning in the proximal end direction with the medical sheet adhering to the second support surface.

(2) The transfer instrument according to the above (1), in which the second support portion may include a tapered portion tapered in the distal end direction between a portion with a maximum width of the second support portion in a width direction orthogonal to an axial direction of the second shaft and a distal end of the second support portion, and when the maximum width is L1 and a length of the tapered portion in the axial direction is L2, L2 may be greater than or equal to 50% of L1.

With this configuration, since L2 is greater than or equal to 50% of L1 (L2≥L1/2), the contact area of the second support surface with the medical sheet at the tapered portion becomes small. It is therefore possible to efficiently prevent, when the second shaft is moved in the proximal end direction along the first shaft after the medical sheet is transferred to the treatment site, the medical sheet from returning in the proximal end direction with the medical sheet adhering to the second support surface.

(3) The transfer instrument according to the above (2), in which side edges of both sides of the tapered portion in the width direction may linearly extend, and a distance between the side edges of both sides in the width direction may decrease toward the distal end of the second support portion.

With this configuration, since the side edges of both sides of the tapered portion in the width direction linearly extend, and the distance between the side edges of both sides decreases toward the distal end of the second support portion, the tapered portion can be formed in an acute triangular shape. This allows, in a simple manner, a reduction in the contact area of the second support surface with the medical sheet at the tapered portion.

(4) The transfer instrument according to the above (2), in which the tapered portion may include a first portion including the portion with the maximum width, a second portion including the distal end of the second support portion, and an intermediate portion between the first portion and the second portion, and side edges of both sides of the intermediate portion in the width direction may extend inward in the width direction.

With this configuration, the tapered portion includes the first portion, the intermediate portion, and the second portion, and the side edges of both sides of the intermediate portion extend inward in the width direction. This allows, in a simple manner, a reduction in the contact area of the second support surface with the medical sheet at the tapered portion.

(5) The transfer instrument according to any one of the above (1) to (4), in which a distal end edge of the second support portion may have an arc shape.

With this configuration, since the distal end edge of the second support portion has an arc shape, it is possible to prevent the distal end edge of the second support portion from damaging the treatment site when the second shaft is moved in the distal end direction along the first shaft. It is further possible to prevent breakage of the medical sheet supported by the second support surface when the medical sheet comes into contact with the distal end edge of the second support portion.

(6) The transfer instrument according to any one of the above (1) to (5), in which the medical sheet may be supported on the second support surface.

With this configuration, it is possible to efficiently transfer, by moving the second shaft along the first shaft with the medical sheet supported on the second support surface, the medical sheet to the treatment site.

(7) The transfer instrument according to any one of the above (1) to (6), in which the second carrier member may further include a pressing portion provided at the distal end of the second shaft, the second support portion may be fixed to the pressing portion, the first support surface may include an upper surface of the first support portion, the second support surface may include a bottom surface of the second support portion, the bottom surface facing the first support surface, the medical sheet may be supported between the first support surface and the second support surface, and the pressing portion may be capable of pressing, in the distal end direction, the medical sheet supported between the first support surface and the second support surface.

With this configuration, since the medical sheet is pressed in the distal end direction by the pressing portion when the second shaft is moved along the first shaft with the medical sheet supported between the first support surface and the second support surface, it is possible to efficiently transfer the medical sheet to the treatment site.

Further, with this configuration, since the medical sheet is supported in the vertical direction by the first support surface and the second support surface, it is possible to transfer the medical sheet to the treatment site with the medical sheet prevented from becoming crinkled.

(8) The transfer instrument according to the above (7), in which the second support portion may include a bent portion obtained by bending a part of the second support portion including a distal end of the second support portion in a direction away from the first support surface.

With this configuration, it is possible to easily cause, by lifting the bent portion in a direction away from the first support surface, the second support portion to curve in the direction away from the first support surface. Accordingly, a gap is formed between the first support surface and the second support surface, so that it is possible to easily place the medical sheet between the first support surface and the second support surface.

(9) The transfer instrument according to the above (7), in which the second support portion may include a projection provided on an upper surface opposite to the second support surface.

With this configuration, it is possible to easily cause, by lifting the projection in a direction away from the first support surface, the second support portion to curve in the direction away from the first support surface. Accordingly, a gap is formed between the first support surface and the second support surface, so that it is possible to easily place the medical sheet between the first support surface and the second support surface.

(10) The transfer instrument according to any one of the above (1) to (9) may further including an outer cylinder including a distal-end opening, in which the first shaft and the second shaft may be movable inside and along the outer cylinder, at a first position where the first shaft and the second shaft are moved in a proximal end direction relative to the outer cylinder to cause the first support portion and the second support portion to retract in the outer cylinder, the first support portion and the second support portion may be retracted in the outer cylinder with the first support portion and the second support portion curved and deformed, and at a second position where the first shaft and the second shaft are moved in the distal end direction relative to the outer cylinder to cause the first support portion and the second support portion to protrude from the distal-end opening, the first support portion and the second support portion may move out from the outer cylinder in the distal end direction to unfold.

With this configuration, the first support portion and the second support portion can be retracted in the outer cylinder in a compact form at the first position. Further, since the second support portion has a shape tapered in the distal end direction, it is possible to effectively prevent the second support portion from becoming crinkled when the second support portion is retracted in the outer cylinder.

(11) The transfer instrument according to the above (10), in which the second support portion may be bent or curved toward the first support portion at a center of the second support portion in a width direction orthogonal to an axial direction of the second shaft, and at the first position, the second support portion may be retracted in the outer cylinder with the second support portion curved and deformed in a round shape around the center that is bent or curved.

With this configuration, the center of the second support portion in the width direction is bent or curved toward the first support portion. This allows the second support portion to be easily curved and deformed at the first position into a round shape around the bent or curved center and retracted in the outer cylinder. As a result, it is possible to effectively prevent the second support portion from becoming crinkled when the second support portion is retracted in the outer cylinder.

(12) The transfer instrument according to the above (11), in which a portion of the second support surface between a portion bent or curved toward the first support portion and a distal end of the second support portion may include a flat surface.

With this configuration, since the portion of the second support surface between the portion bent or curved toward the first support portion and the distal end of the second support portion includes the flat surface, the medical sheet is easily supported by the flat surface.

According to the disclosed transfer instruments and methods, it is possible to efficiently transfer, by moving the second shaft along the first shaft with the medical sheet supported by the first support surface and the second support surface, the medical sheet to the treatment site.

Further, since the second support portion has a shape tapered in the distal end direction, the contact area of the second support surface with the medical sheet becomes smaller toward the distal end, and frictional resistance between the medical sheet and the second support surface decreases accordingly. It is therefore possible to prevent, when the second shaft is moved in the proximal end direction along the first shaft after the medical sheet is transferred to the treatment site, the medical sheet from returning in the proximal end direction with the medical sheet adhering to the second support surface.

According to another aspect, a transfer instrument for transferring a medical sheet to a treatment site of a living body comprises an outer cylinder having a through hole that extends throughout the outer cylinder from a distal-end opening at a distal end of the outer cylinder to a proximal-end opening at a proximal end of the outer cylinder, a first carrier member, and a second carrier member. The first carrier member includes a first shaft and a first support portion, with the first shaft extending in an extending direction and having a distal end portion at which is fixed the first support portion so that the first shaft and the first support portion move together, and with the first support portion extending in a distal direction away from the first shaft and having a first support surface configured to support the medical sheet. The second carrier member includes a second shaft and a second support portion, with the second shaft extending in the extending direction and having a distal end portion at which is fixed the second support portion so that the second shaft and the second support portion move together, and with the second support portion extending in a distal direction away from the second shaft and having a second support surface configured to support the medical sheet. The first shaft and the second shaft are positionable in the through hole in the outer cylinder and are: i) axially movable in a proximal direction relative to the outer cylinder to move the first support portion and the second support portion from an extended position in which the first support portion and the second support portion are distal of the distal-end opening of the outer cylinder while supporting the medical sheet to a retracted position in which the first support portion and the second support portion are retracted into the through hole in the outer cylinder together with the medical sheet; and ii) axially movable in a distal direction relative to the outer cylinder to move the first support portion and the second support portion from the retracted position to the extended position. The second support portion has a width orthogonal to the extending direction, and the second support portion, in plan view when in the extended position, has a tapered portion that has a tapering shape in which the width of the tapered portion of the second support portion narrows in the distal direction.

Another aspect involves a method comprising: moving a first support portion and a second support portion support portion toward a medical sheet. The first support portion is fixed to a distal end portion of a first shaft and extends in a distal direction away from the first shaft, with the first support portion having a first support surface. The second support portion is fixed to a distal end portion of a second shaft that extends in an extending direction, with the second support portion extending in a distal direction away from the second shaft. The second support portion is positioned in overlying relation to the first support portion, the second support portion has a width orthogonal to the extending direction, and the second support portion has a tapered portion that has a tapering shape in which the width of the tapered portion of the second support portion narrows in the distal direction. The second support portion, inclusive of the tapered portion of the second support portion, has a second support surface. The method additionally includes positioning the medical sheet so that the medical sheet is supported by the first support surface and the second support surface, inclusive of the second support surface of the tapered portion.

### BRIEF DESCRIPTION OF THE DRAWINGS

FIG. 1 is a perspective view of a transfer instrument according to a first embodiment.

FIG. 2 is an exploded perspective view of the transfer instrument illustrated in FIG. 1.

FIG. 3 is a plan view of a distal end of the transfer instrument illustrated in FIG. 1.

FIG. 4 is a longitudinal cross-sectional view taken along line IV-IV in FIG. 3.

FIG. 5 is a transverse cross-sectional view taken along line V-V in FIG. 3.

FIG. 6 is a flowchart illustrating a procedure of a transfer method for transferring a medical sheet using the transfer instrument illustrated in FIG. 1.

FIG. 7 is a first explanatory diagram illustrating how the medical sheet is placed in a sheet placing process.

FIG. 8 is a second explanatory diagram illustrating how the medical sheet is placed in the sheet placing process.

FIG. 9 is an explanatory diagram of a retracting process.

FIG. 10 is a transverse cross-sectional view taken along line X-X in FIG. 9.

FIG. 11 is an explanatory diagram of a positioning process.

FIG. 12 is an explanatory diagram of an unfolding process;

FIG. 13 is an explanatory diagram of a moving process;

FIG. 14 is an explanatory diagram of a withdrawing process.

FIG. 15 is a plan view of a distal end of a transfer instrument according to a second embodiment.

FIG. 16 is a perspective view of a distal end of a transfer instrument according to a third embodiment.

FIG. 17 is a perspective view of a distal end of a transfer instrument according to a fourth embodiment.

FIG. 18 is a transverse cross-sectional view of the transfer instrument illustrated in FIG. 17.

FIG. 19 is an explanatory diagram of a moving process.

FIG. 20 is a perspective view of a distal end of a transfer instrument according to a fifth embodiment.

### DETAILED DESCRIPTION

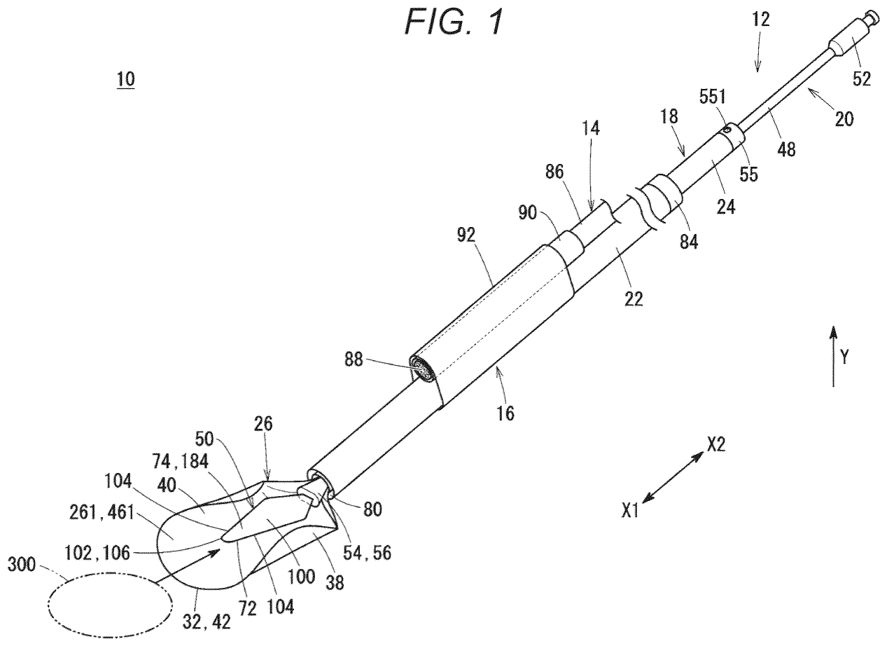

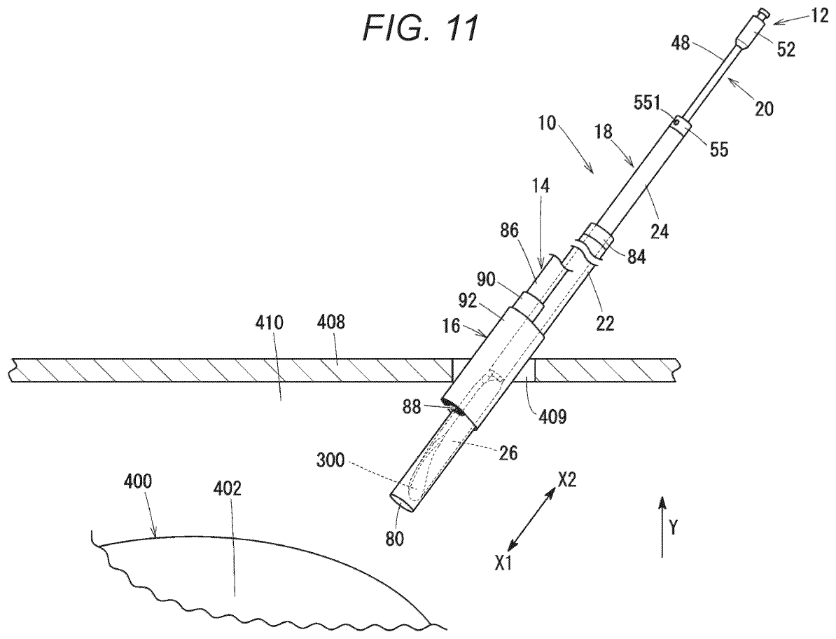

FIG. 1 is a perspective view of a transfer instrument 10 according to a first embodiment. The transfer instrument 10 is a medical instrument for transferring a medical sheet 300 to a treatment site of a living body. The transfer instrument 10 is used for, for example, the treatment of severe heart failure caused by ischemic heart disease. In this case, the medical sheet 300 is transplanted to a recipient site 402 of a heart 400 (the treatment site of the living body) (see FIGS. 11 to 14). The transfer instrument 10 is capable of attaching a plurality of the medical sheets 300 to the recipient site 402.

Examples of such a medical sheet 300 include pharmaceutical products or regenerative medicine products for medical use, a medical instrument, and the like. The medical sheet 300 is formed in a sheet shape such as a film shape or a membrane shape (gel object). Fibrin or the like may be applied to the medical sheet 300 for reinforcement. Examples of the regenerative medicine products including cells include a cell sheet (sheet-shaped cell culture), a spheroid, and the like. It is possible to form the cell sheet by culturing autologous cells or allogenic cells. The cells constituting the cell sheet include, for example, somatic stem cells (adult stem cells), mesenchymal stem cells, or iPS cells (induced pluripotent stem cells)-derived cardiomyocytes. Examples of the somatic stem cells preferably include skeletal myoblast cells (myoblast cells).

The medical sheet 300 may contain a tissue adhesive, a local anesthetic, or the like. The medical sheet 300 has a thickness of, for example, about 100 μm, and has a diameter of, for example, about 60 mm. thickness and the diameter (size) of the medical sheet 300 are not limited these values and can be set as desired.

The medical sheet 300 may be a sheet to be transplanted to an organ (for example, lung, liver, pancreas, kidney, small intestine, esophagus, or the like) other than the heart 400. Further, the medical sheet 300 may be, for example, an anti-adhesion sheet as long as the sheet is for medical use.

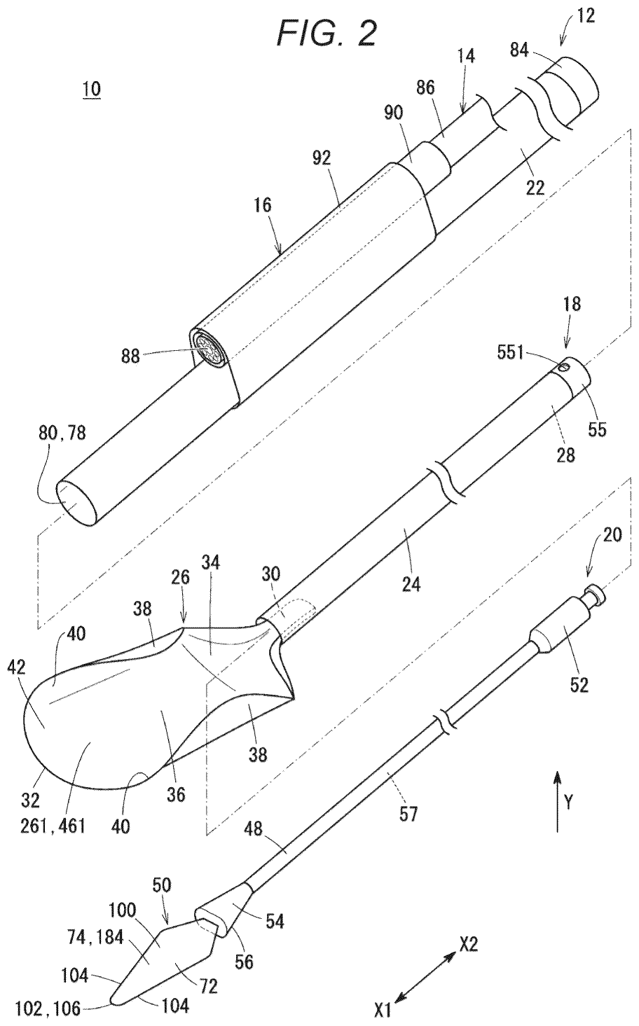

As illustrated in FIGS. 1 and 2, the transfer instrument 10 includes an instrument body 12, an endoscope 14, and a fixing member 16. The instrument body 12 includes a first carrier member 18, a second carrier member 20, and an outer cylinder 22. The transfer instrument 10 is not limited to a configuration including the endoscope 14.

In FIG. 2, the first carrier member 18 includes a first shaft 24 and a first support portion 26.

The first shaft 24 is a tubular body (in the first embodiment, a circular tube member) having a first lumen 28. The first lumen 28 opens at a distal end (end in a direction of arrow X1) of the first shaft 24 and opens at a proximal end (end in a direction of arrow X2) of the first shaft 24. The first shaft 24 has an airtight valve 55 provided at its proximal end with the valve 55 in close contact with an outer circumferential surface of the second shaft 48. The valve 55 has a marker 551 provided on its outer circumferential surface. When the transfer instrument 10 is used, the marker 551 is visible to the user. The first shaft 24 is not limited to a tubular body and may be a body other than a tubular body.

The first shaft 24 extends in an axial direction of the outer cylinder 22 and is disposed inside the outer cylinder 22 in a manner as to be movable along the axial direction. The first shaft 24 includes, for example, a resin material. Examples of the constituent material from which the first shaft 24 may be fabricated include, but not particularly limited to, polyethylene, polypropylene, fluororesin, polyethylene terephthalate, polymethyl methacrylate, a polyamide resin, polystyrene, polycarbonate, polyimide, polyetherimide, polyetheretherketone, polyvinyl chloride, an ABS resin, a polyamide elastomer, and a polyester elastomer. The first shaft 24 may include a metal material.

The first shaft 24 may be flexible. The first shaft 24 may have a flexible tube portion capable of maintaining a bent shape. In this case, the first shaft 24 can be bent into an any desired shape in a body cavity and can maintain the bent shape.

As illustrated in FIGS. 2 to 4, the first support portion 26 is attached to the distal end of the first shaft 24. The first support portion 26 includes or is fabricated from, for example, a resin material. The first support portion 26 can hold the medical sheet 300. A flexible resin sheet member (film member) is bent into a predetermined form to form the first support portion 26. Alternatively, the sheet member is shaped into a predetermined form by a sheet forming die to form the first support portion 26. It is preferable that the sheet member have, but not particularly limited to, a thickness of, for example, 100 μm or more and 200 μm or less. The first support portion 26 includes a first joint 30 and a first support body 32.

The constituent material from which the first support portion 26 may be fabricated preferably has transparency, and examples of the constituent material include, but are not particularly limited to, polyethylene, polycarbonate, polyamide, polystyrene, polypropylene, polyacetal resin, polyimide, polyetherimide, polyetheretherketone, polyethylene terephthalate, and fluororesin. Further, the first support portion 26 may have a mesh shape.

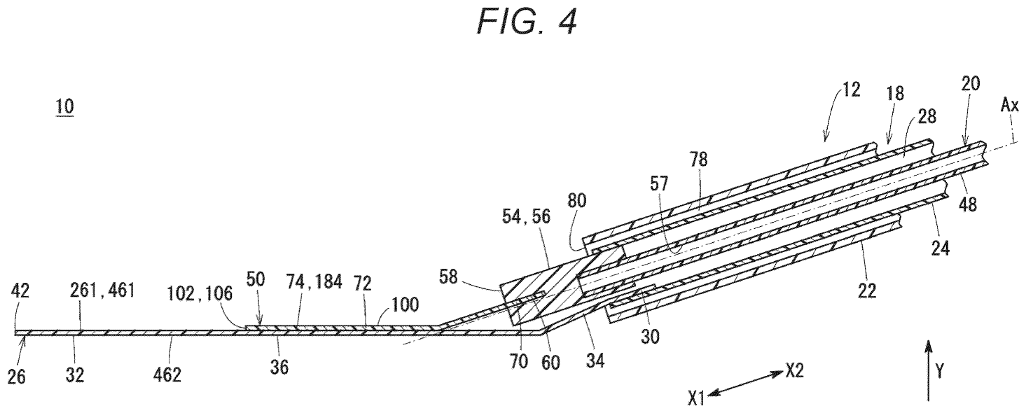

In FIG. 4, the first joint 30 is bonded to an inner circumferential surface of the distal end of the first shaft 24 with an adhesive. Examples of the adhesive include, but not particularly limited to, a UV adhesive, a hot-melt adhesive, and an instant adhesive (for example, cyanoacrylate-based instant adhesive). The first joint 30 may be thermally fused to the inner circumferential surface of the first shaft 24. The first support portion 26 may be detachable from the distal end of the first shaft 24.

As illustrated in FIG. 2, the first support body 32 extends in a distal end direction from first joint 30. The first support body 32 includes a proximal-end support portion 34, an intermediate support portion 36, a pair of first protrusions 38, a pair of second protrusions 40, and a distal-end support portion 42. The first support body 32 includes a first support surface 261. The marker 551 of the first shaft 24 is disposed so as to face upward when the first support surface 261 faces upward. That is, the marker 551 allows the user to confirm the direction of the first support surface 261 of the first support portion 26.

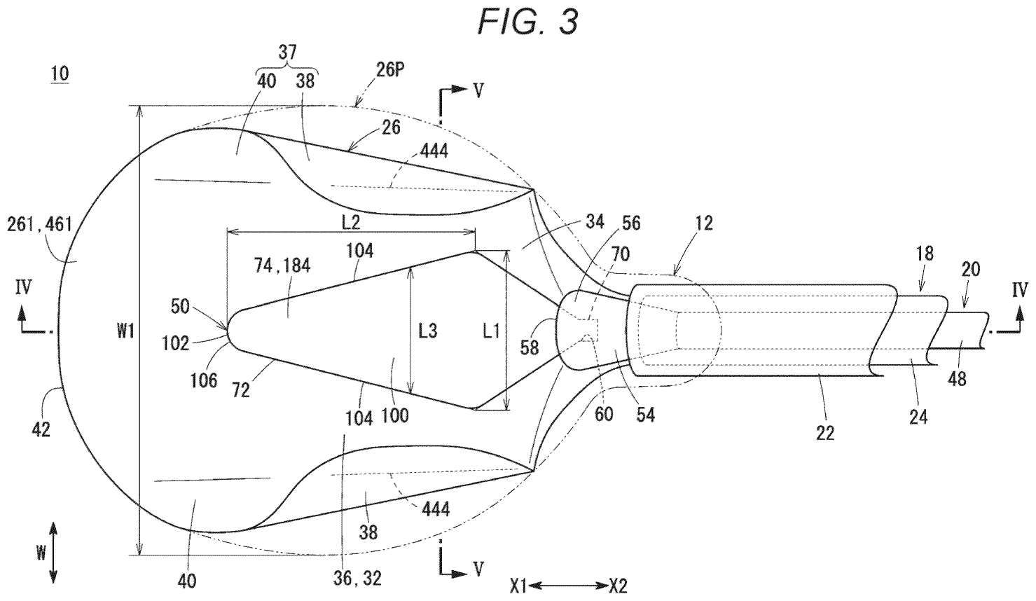

As illustrated in FIG. 3, the proximal-end support portion 34 is formed to be wider in its extending direction. In other words, both sides of the proximal-end support portion 34 in a width direction (a direction of arrow W orthogonal to the direction of arrow X1, the direction of arrow X2, and a direction of arrow Y) are tapered toward the first joint 30 (see FIG. 2). The intermediate support portion 36 is formed in a tapered shape so as to be gradually narrower in width from the distal end toward the proximal end (in the direction of arrow X2).

The distal-end support portion 42 is connected to a distal end of the intermediate support portion 36 and distal ends of the pair of second protrusions 40. The distal-end support portion 42 protrudes in an arc shape in the distal end direction (the direction of arrow X1). That is, when viewed from a direction perpendicular to the first support surface 261 illustrated in FIG. 3, the distal-end support portion 42 of the first support portion 26 has an arc shape connecting the pair of second protrusions 40.

As illustrated in FIG. 4, the proximal-end support portion 34 extends roughly along an axis Ax of the first shaft 24 from a distal end of the first joint 30 in the distal end direction (the direction of arrow X1). The intermediate support portion 36 intersects with the axis Ax of the first shaft 24 and extends from a distal end of the proximal-end support portion 34 toward the distal end of the first support portion 26 (in the direction of arrow X1).

In FIGS. 2 and 3, the pair of first protrusions 38 protrude upward (in the direction of arrow Y) from both sides of the intermediate support portion 36 in the width direction (the direction of arrow W) orthogonal to a direction in which the first shaft 24 moves and inward in the width direction of the intermediate support portion 36. The pair of first protrusions 38 are connected to the proximal-end support portion 34. Each first protrusion 38 has a convex shape extending in a direction away from the first support surface 261.

As illustrated in FIG. 3, the pair of second protrusions 40 are connected to distal ends of the pair of first protrusions 38. The pair of second protrusions 40 protrude upward from both sides of the intermediate support portion 36 in the width direction and outward in the width direction of the intermediate support portion 36. Each second protrusion 40 is formed with a smaller curvature than the first protrusion 38. The second protrusions 40 are lower in protrusion height relative to the first support surface 261 than the first protrusions 38.

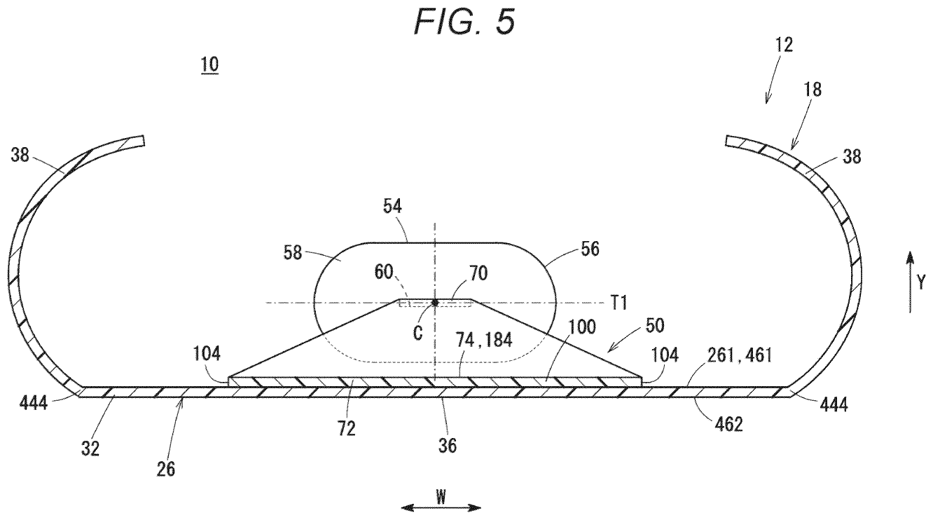

Each of the pair of first protrusions 38 has one of a pair of bent portions 444. Each of the pair of bent portions 444 causes a corresponding one of the pair of first protrusions 38 to bend from the first support surface 261 (intermediate support portion 36) of the first support portion 26 (see FIG. 5).

The first support body 32 includes a front surface 461 that faces upward (in the direction of arrow Y) and includes the first support surface 261, and a back surface 462 that is a surface opposite to the front surface 461. The first support surface 261 includes a flat surface continuous over an upper surface of the proximal-end support portion 34 and upper surfaces of the intermediate support portion 36 and the distal-end support portion 42. A lubricant may be applied to the first support surface 261 so as to allow a second support portion 50 (to be described later) of the second carrier member 20 to smoothly slide on the first support surface 261.

As illustrated in FIG. 2, the second carrier member 20 includes a second shaft 48, the second support portion 50, and a hub 52.

The second shaft 48 is a tubular body (in the first embodiment, a circular tube member) having a second lumen 57. The second shaft 48 is longer in the axial direction than the first shaft 24. The second shaft 48 is inserted into or positioned in the first lumen 28 of the first shaft 24 (see FIGS. 1 and 4). In other words, a distal end of the second shaft 48 protrudes in the distal end direction (the direction of arrow X1) relative to the distal-end opening of the first shaft 24. A proximal end of the second shaft 48 protrudes in the proximal end direction (the direction of arrow X2) relative to the proximal-end opening of the first shaft 24 (see FIG. 1). The second shaft 48 is provided extending along the first shaft 24 so as to be movable along the first shaft 24. The second shaft 48 is not limited to a tubular body and may be a body other than a tubular body.

The second shaft 48 is configured to follow the shape of the first support portion 26. As the constituent material from which the second shaft 48 may be fabricated, for example, a material more flexible than the constituent material of the first shaft 24 is selected. Specifically, examples of the constituent material of the second shaft 48 include a polyamide elastomer, a polyester elastomer, a polyurethane elastomer, polyvinyl chloride, polybutadiene, a silicone rubber, and a metal coil (including a composite with a resin). The second shaft 48 is flexible.

As illustrated in FIG. 4, the second shaft 48 includes a carrier holding portion 54 (pressing portion) and a pressure-application portion 56 that is the distal end of the second shaft 48. The pressure-application portion 56 includes an elastic body such as an elastomer member. The pressure-application portion 56 presses the first support portion 26 against an inner surface of the outer cylinder 22 with the first support portion 26 retracted in the outer cylinder 22.

A distal end of the carrier holding portion 54 includes a pressing surface 58. The carrier holding portion 54 can cause the pressing surface 58 to press an outer edge surface of the medical sheet 300 supported by the first support portion 26 in the distal end direction (the direction of arrow X1). In the first embodiment, the pressure-application portion 56 is provided with the carrier holding portion 54 that supports the second support portion 50. The carrier holding portion 54 includes the pressing surface 58 and an attachment hole 60.

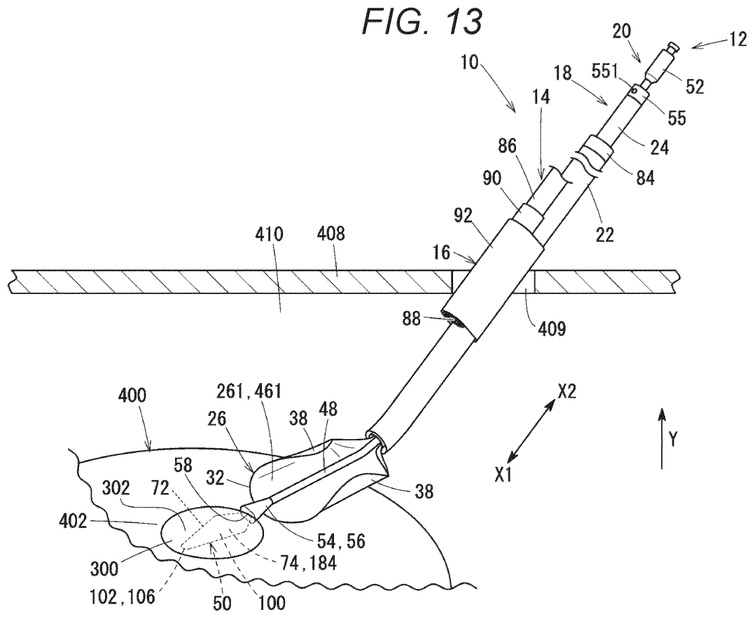

In FIG. 4, the pressing surface 58 is provided on a distal end surface of the carrier holding portion 54. The attachment hole 60 opens at the pressing surface 58. The second support portion 50 is attached to the pressing surface 58. The pressing surface 58 presses the outer edge surface of the medical sheet 300 in the distal end direction (the direction of arrow X1) (see FIG. 13).

In FIGS. 2 to 4, the second support portion 50 is a flexible sheet. The second support portion 50 includes a second joint 70 and a second support body 72. The second joint 70 is provided at a proximal end of the second support portion 50. The second joint 70 is provided at a proximal end of the second support body 72. The second joint 70 is inserted into or positioned in the attachment hole 60 of the carrier holding portion 54, and is bonded, for example. The second joint 70 may be joined to the attachment hole 60 of the carrier holding portion 54 by a suitable joining method other than adhesion. The second support portion 50 may be integrally formed with the carrier holding portion 54.

The second support body 72 extends in the distal end direction (the direction of arrow X1) from the second joint 70. The second support body 72 extending from the second joint 70 is shorter in the extending direction than the first support body 32 extending from the first joint 30. The second support body 72 is provided with, on its upper surface 184, a second support surface 74 on which the medical sheet 300 is placed. The second support surface 74 includes a flat surface. The second support body 72 is smaller than the first support body 32. That is, the second support surface 74 is smaller in area than the first support surface 261.

In FIG. 2, the hub 52 is attached to the proximal end of the second shaft 48.

In FIGS. 1 and 2, the outer cylinder 22 is a cylindrical member having a lumen 78. The lumen 78 has a distal-end opening 80 that opens at a distal end (end in the direction of arrow X1) of the outer cylinder 22. The lumen 78 opens at a proximal end (end in the direction of arrow X2) of the outer cylinder 22. The outer cylinder 22 is flexible. Examples of the constituent material from which the outer cylinder 22 may be fabricated are the same as the examples of the constituent material of the first shaft 24 described above.

The first shaft 24 is inserted into or positioned in the lumen 78 of the outer cylinder 22. The outer cylinder 22 is shorter in length along the axial direction than the first shaft 24. The outer cylinder 22 is provided with, at its proximal end, an airtight valve 84 that is in close contact with an outer circumferential surface of the first shaft 24.

In FIGS. 2 and 4, a distal end surface of the outer cylinder 22 extends orthogonal to the axial direction of the outer cylinder 22.

As illustrated in FIG. 2, the endoscope 14 includes a long or elongated endoscope main body 86. A distal end of the endoscope main body 86 is fixed to an outer circumferential surface of the outer cylinder 22 by the fixing member 16 (see FIG. 1). An objective lens 88 provided on a distal end surface of the endoscope main body 86 is oriented toward the distal end of the outer cylinder 22 (in the direction of arrow X1). The distal end of the endoscope main body 86 is fixed to an intermediate portion of the outer cylinder 22 in the axial direction. The distal end of the endoscope main body 86 may be fixed to the distal end of the outer cylinder 22.

The fixing member 16 includes, for example, a fixing cylinder 90 and a fixing tube 92. The fixing cylinder 90 includes, for example, a hard resin material. The endoscope main body 86 can be inserted into or positioned in a lumen of the fixing cylinder 90. The fixing cylinder 90 is disposed along a longitudinal direction of the outer cylinder 22. The fixing tube 92 is a tube for fixing the fixing cylinder 90 at a predetermined position of the outer cylinder 22. The fixing tube 92 is, for example, a heat-shrink tube. The manner in which the distal end of the endoscope main body 86 is fixed to the outer cylinder 22 may be determined as desired.

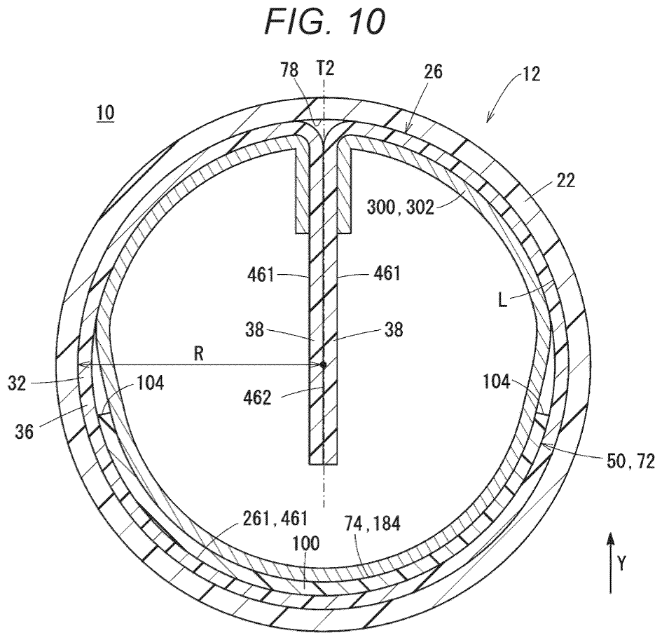

As illustrated in FIG. 3, a portion including both the first protrusion 38 and the second protrusion 40 of the first support portion 26 is hereinafter referred to as “protrusion 37”. Therefore, the protrusion 37 is provided as a pair on both sides of the first support portion 26 in the width direction. In FIG. 3, the direction (W direction) orthogonal to the axial direction of the first shaft 24 refers to the width direction of the first support portion 26. The W direction also refers to a width direction of other components (the outer cylinder 22 and the like). In the first embodiment, the first support portion 26 includes the pair of protrusions 37, and W1 in FIG. 3 denotes a width of the widest portion of the first support portion 26 when the first support portion 26 is unfolded to be flat. That is, the width W1 of the first support portion 26 is the maximum width of a first support portion 26P in a flat state before the pair of protrusions 37 are formed (before folded). The width W1 of the first support portion 26 is larger than an inner circumferential length L of the outer cylinder 22 (see FIG. 10). In FIG. 10, the inner circumferential length L of the outer cylinder 22 is a length of an inner circumferential surface along a circumferential direction of the outer cylinder 22 (L=2πR). R denotes the radius of the lumen 78 of the outer cylinder 22.

As illustrated in FIG. 5, in the cross section orthogonal to the axis of the second shaft 48, the carrier holding portion 54 has a line-symmetrical shape with respect to an imaginary line T1 passing through a center line C of the carrier holding portion 54 and parallel to the width direction (the direction of arrow W) of the carrier holding portion 54. In other words, the carrier holding portion 54 has a symmetrical shape in a vertical direction with respect to the imaginary line T1. As illustrated in FIG. 5, the carrier holding portion 54 has a flattened cross-sectional shape. The carrier holding portion 54 is not limited to a configuration formed in a flattened cross-sectional shape, and may be formed in, for example, a circular cross-sectional shape, an elliptical cross-sectional shape, a square cross-sectional shape, a rectangular cross-sectional shape, or the like.

The attachment hole 60 is disposed on the center line C of the carrier holding portion 54 on the pressing surface 58. The attachment hole 60 has a slit shape extending parallel to the width direction of the carrier holding portion 54 from the center line C. The attachment hole 60 has a symmetrical shape in the width direction with respect to the center line C. The attachment hole 60 extends in the axial direction from the pressing surface 58 (see FIG. 4). As described above, the second joint 70 of the second support portion 50 is inserted into and connected to the attachment hole 60.

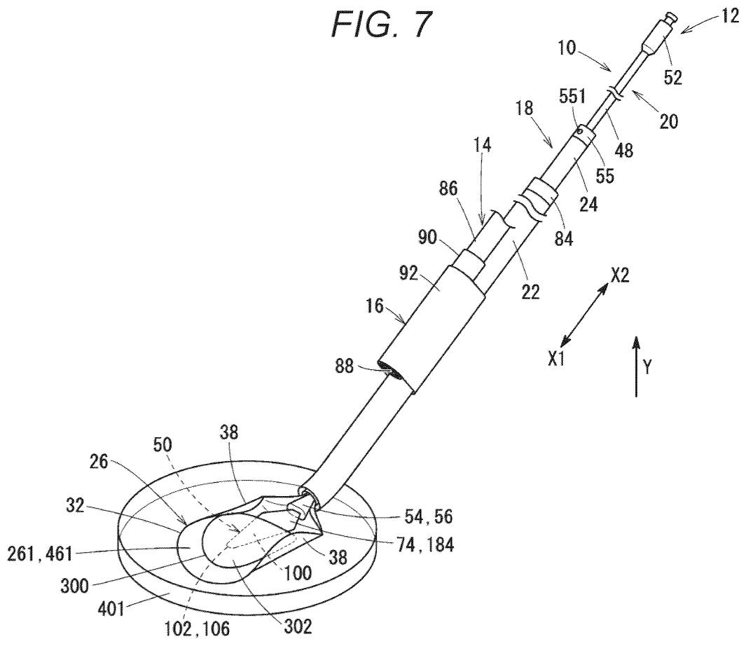

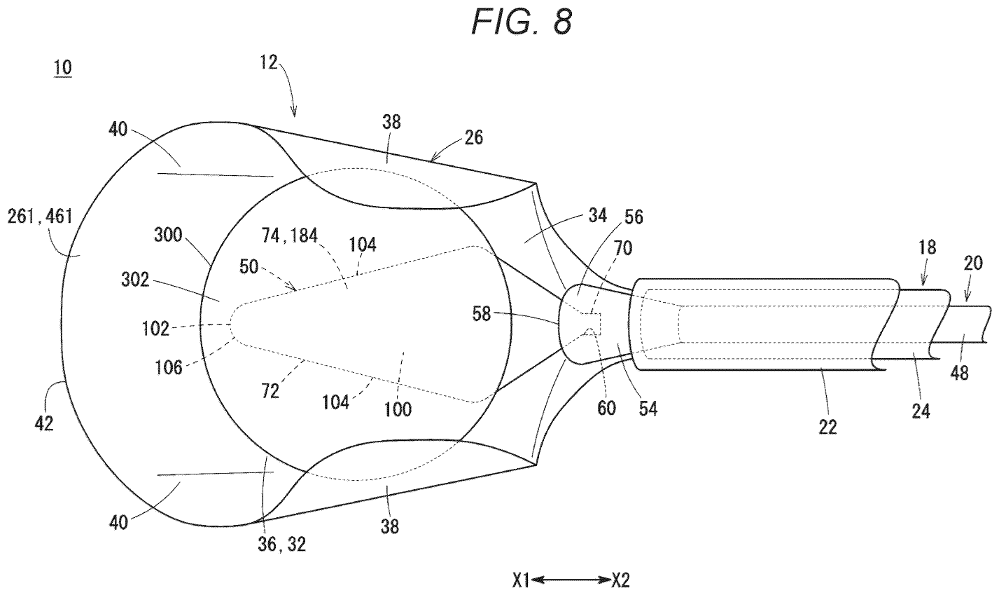

The medical sheet 300 is supported by the first support surface 261 and the second support surface 74 (see FIGS. 7 and 8). Specifically, the medical sheet 300 is placed on the first support surface 261 and the second support surface 74. When the medical sheet 300 is placed on the second support surface 74, a portion of the medical sheet 300 that does not fit within the second support surface 74 is supported by the first support surface 261.

As illustrated in FIGS. 1 to 3, the second support portion 50 is tapered in the distal end direction. Specifically, the second support portion 50 is flared in the width direction (the direction of arrow W) from the second joint 70 in the distal end direction and is then tapered in the width direction from the portion with the maximum width L1 of the second support portion 50 in the distal end direction. The second support portion 50 includes a tapered portion 100. The tapered portion 100 is a portion of the second support portion 50 between the portion with the maximum width L1 and a distal end 102 of the second support portion 50.

When a length of the tapered portion 100 in the axial direction of the second shaft 48 is denoted by L2, L2 is greater than or equal to 50% of L1 (L2≥L1/2). L2 is preferably equal to or greater than L1 (L2≥L1). L2 is more preferably greater than or equal to 150% of L1 (L2≥1.5×L1).

The tapered portion 100 is formed in a triangular shape with the distal end 102 of the second support portion 50 as an acute angle. Side edges 104 of both sides of the tapered portion 100 in the width direction linearly extend. A distance L3 between the two side edges 104 in the width direction decreases toward the distal end 102 of the second support portion 50.

A distal end edge 106 of the second support portion 50 including the distal end 102 of the second support portion 50 has an arc shape. The distal end edge 106 of the second support portion 50 is not limited to a configuration having an arc shape. The distal end edge 106 of the second support portion 50 may have any shape as long as the medical sheet 300 does not break when the distal end edge 106 comes into contact with the medical sheet 300.

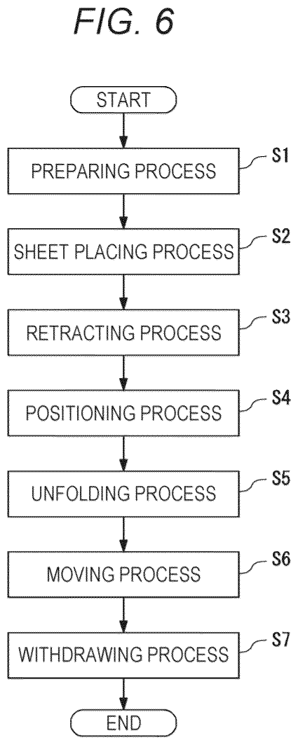

Next, a transfer method for transferring the medical sheet 300 to a treatment site of a living body will be described. Specifically, as illustrated in FIGS. 11 to 14, the transfer method for transferring the medical sheet 300 to the recipient site 402 of the heart 400 (the treatment site of the living body) during thoracoscopic surgery will be described. As illustrated in FIG. 6, the transfer method according to the first embodiment includes a preparing process, a sheet placing process, a retracting process, a positioning process, an unfolding process, a moving process, and a withdrawing process.

First, in the preparing process (step S1), the transfer instrument 10 according to the first embodiment described above is prepared. The following description will be given on the assumption that a state as illustrated in FIG. 1 is an initial state of the transfer instrument 10. In the initial state, the first shaft 24 and the second shaft 48 are moved in the distal end direction (the direction of arrow X1) relative to the outer cylinder 22 so as to be at a protruding position (second position) where the first support portion 26 and the second support portion 50 protrude from the distal-end opening 80 of the outer cylinder 22 in the distal end direction. The first and second support portions 26 and 50 are each unfolded when moving out of the outer cylinder 22 in the distal end direction, and the second support portion 50 is positioned on the first support surface 261 of the first support portion 26. That is, the second support portion 50 is positioned at a retracted position where the second support portion 50 is placed on the first support surface 261 of the first support portion 26. At this time, a proximal end of the carrier holding portion 54 is in the first lumen 28 of the first shaft 24.

Next, in the sheet placing process (step S2), as illustrated in FIGS. 7 and 8, the medical sheet 300 placed on or located in a Petri dish 401 is placed on the second support surface 74. The medical sheet 300 protrudes outward from the second support portion 50 with the medical sheet 300 placed on the second support surface 74. The first support surface 261 supports an overhanging portion 302 of the medical sheet 300 that sticks out over the second support portion 50.

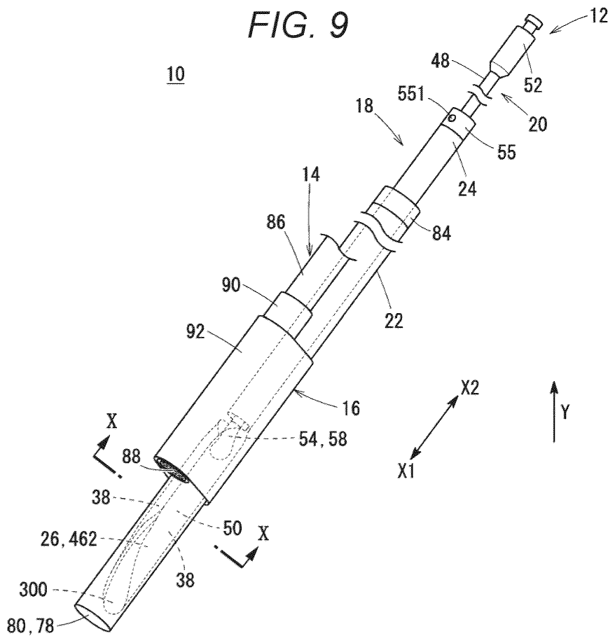

Subsequently, in the retracting process (step S3 in FIG. 6), as illustrated in FIG. 9, the medical sheet 300 is retracted in the outer cylinder 22 together with the first support portion 26 and the second support portion 50 (see FIG. 8) at a retracted position (first position). Specifically, the first shaft 24 of the first carrier member 18 and the second shaft 48 of the second carrier member 20 are moved together in the proximal end direction (the direction of arrow X2) relative to the outer cylinder 22.

Then, the proximal-end support portion 34 (see FIG. 2) of the first support portion 26 is pulled in the proximal end direction through the distal-end opening 80 of the outer cylinder 22. At this time, when both the tapered sides of the proximal-end support portion 34 come into contact with the distal-end opening 80 of the outer cylinder 22, a force acts on the proximal-end support portion 34 to cause the proximal-end support portion 34 to curl along the circumferential direction of the outer cylinder 22. Therefore, the proximal-end support portion 34 is smoothly pulled into the outer cylinder 22 while curling. At this time, the first support portion 26 is retracted in the outer cylinder 22 while curling in a conical shape such that the distal end side of the first support portion 26 becomes larger in diameter than the proximal-end support portion 34.

When the proximal-end support portion 34 of the first support portion 26 becomes deformed, a force acts on the intermediate support portion 36 of the first support portion 26 to cause the intermediate support portion 36 to curl along the circumferential direction of the outer cylinder 22, so that the intermediate support portion 36 (see FIG. 2) is pulled into the outer cylinder 22 while curling. At this time, the intermediate support portion 36 becomes deformed into a cylindrical shape along the inner surface of the outer cylinder 22. Each of the pair of first protrusions 38 is curved such that the front surface 461 of the first support portion 26 faces inward and the back surface 462 of the first support portion 26 faces outward. As illustrated in FIG. 10, the back surfaces 462 of both sides of the first support portion 26 in the width direction come into contact with each other on an imaginary line T2 extending orthogonal to the center axis of the outer cylinder 22. One of the first protrusions 38 and the other first protrusion 38 come into contact with each other and are retracted downward (toward the first support surface 261, the front surface 461).

Accordingly, the back surface 462 of the first support portion 26 is curved and is in close contact with the inner surface of the outer cylinder 22, each of the first protrusions 38 is further curved to fold back toward the center of the outer cylinder 22, and a free end of each of the first protrusions 38 is positioned below the center axis of the outer cylinder 22. That is, the first support portion 26 is curved in a heart shape along the inner surface of the outer cylinder 22.

The heart shape refers to a roughly round shape including two convex shapes, a convex shape on one side and a convex shape on the other side. In a case where the heart shape is formed in the lumen 78 of the tubular body (outer cylinder 22), the two convex shapes protruding toward the opposite sides along the inner surface of the tubular body come close to each other so as to bring their respective circumferential surfaces into partial contact with each other, so that the entire contour becomes a roughly round shape along the inner surface of the tubular body (refer to the shape of the first support portion 26 in FIG. 10).

Along with the curved deformation of the first support portion 26, the second support portion 50 is similarly curved and deformed along the first support portion 26 inside the first support portion 26 (on the front surface 461 side). Along with the curved deformation of the first support portion 26 and the second support portion 50, the medical sheet 300 is deformed into a shape corresponding to the shape of the first support body 32 and the shape of the second support body 72, and the medical sheet 300 is retracted in the outer cylinder 22 accordingly.

As illustrated in FIG. 9, when the first support portion 26 is entirely inserted into or positioned in the outer cylinder 22, the retracting process is complete. At the completion of the retracting process, the first support portion 26 may partially protrude from the distal-end opening 80 of the outer cylinder 22. In this case, the state where the first support portion 26 partially protrudes from the distal-end opening 80 of the outer cylinder 22 corresponds to the first position of the first support portion 26.

Subsequently, in the positioning process (step S4 in FIG. 6), as illustrated in FIG. 11, the transfer instrument 10 is inserted into a chest cavity 410 through an incision 409 in a chest 408. At this time, the distal end of the transfer instrument 10 is positioned near the recipient site 402 of the heart 400, and the distal end of the endoscope 14 is positioned in the chest cavity 410. Note that before the transfer instrument 10 is inserted into the chest cavity 410, a liquid supply instrument (not illustrated) may be connected to a connection port of the hub 52 to introduce a liquid (for example, a physiological saline solution).

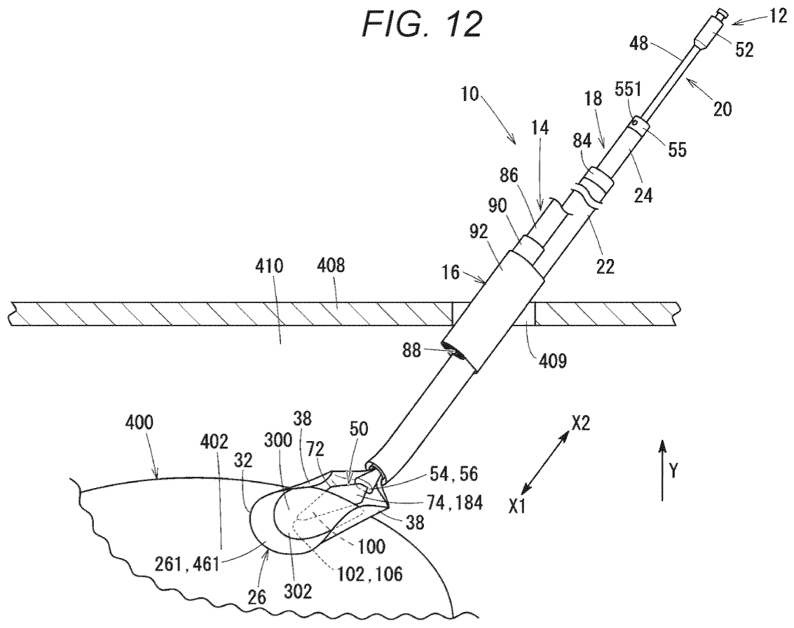

Subsequently, in the unfolding process (step S5 in FIG. 6), as illustrated in FIG. 12, the first support portion 26, the second support portion 50, and the medical sheet 300 are unfolded. Specifically, in the unfolding process, with the first shaft 24 gripped, the first shaft 24 is moved in the distal end direction (the direction of arrow X1) relative to the outer cylinder 22. Accordingly, the valve 55 of the first shaft 24 causes the second shaft 48 to move together with the first shaft 24 in the distal end direction (the direction of arrow X1). Then, the first support portion 26 that has moved out of the distal-end opening 80 of the outer cylinder 22 returns to the original shape due to its restoring force. At the second position where the first support portion 26 is unfolded, the second support portion 50 unrolls flat.

In the unfolding process, of the second carrier member 20, the second support surface 74 on which the medical sheet 300 is placed is entirely positioned on the first support surface 261. At this time, the medical sheet 300 is supported by the first support surface 261 and the second support surface 74. It is therefore possible to prevent the overhanging portion 302 of the medical sheet 300 from becoming crinkled before the medical sheet 300 is transferred to the recipient site 402 of the heart 400.

Next, in the moving process (step S6 in FIG. 6), as illustrated in FIG. 13, the second carrier member 20 is moved in the distal end direction (the direction of arrow X1) relative to the first carrier member 18, so that the second support portion 50 on which the medical sheet 300 is placed is moved from the retracted position to an advanced position, and the second support portion 50 protrudes in the distal end direction (the direction of arrow X1) relative to the distal end of the first support portion 26. Specifically, in the moving process, the second shaft 48 is moved in the distal end direction relative to the first shaft 24.

Accordingly, the second support portion 50 moves in the distal end direction (the direction of arrow X1) relative to the first support portion 26. At this time, when the distal end surface (pressing surface 58) of the carrier holding portion 54 (pressure-application portion 56) presses the outer edge surface of the medical sheet 300 in the distal end direction, the medical sheet 300 is entirely positioned further than the first support portion 26 in the distal end direction. In this moving process, the medical sheet 300 is moved to above the recipient site 402 of the heart 400 to bring the overhanging portion 302 of the medical sheet 300 into contact with the recipient site 402.

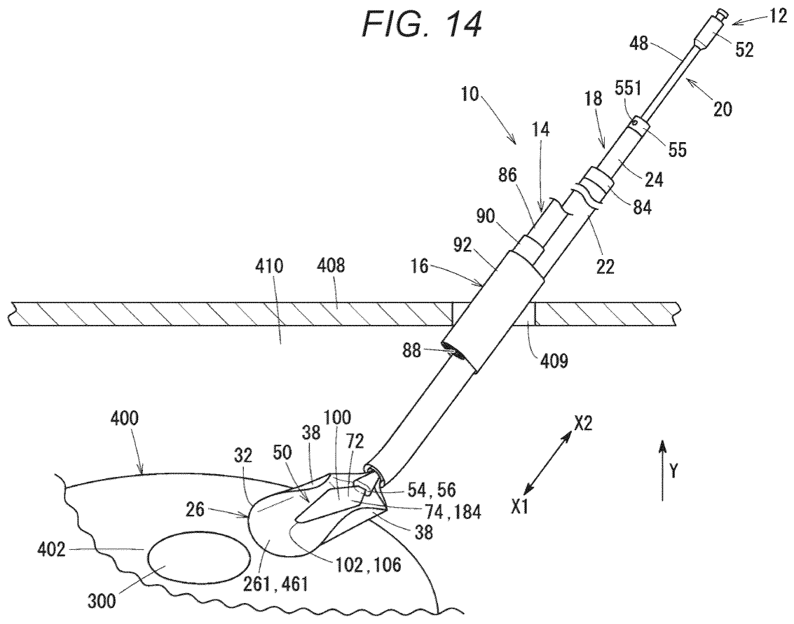

Subsequently, in the withdrawing process (step S7 in FIG. 6), as illustrated in FIG. 14, the second carrier member 20 is moved from the second position to the first position. Since the tapered portion 100 of the second support portion 50 has a shape tapered in the distal end direction, a contact area of the second support surface 74 with the medical sheet 300 becomes smaller toward the distal end. This configuration makes frictional resistance between the medical sheet 300 and the second support surface 74 small. It is therefore possible to easily withdraw, when the second carrier member 20 is moved from the second position to the first position, the second support portion 50 from between the recipient site 402 and the medical sheet 300. Accordingly, the medical sheet 300 entirely comes into contact with the surface of the recipient site 402. As a result, even when the second carrier member 20 is moved from the second position to the first position, the medical sheet 300 does not return in the proximal end direction. This is the end of the transfer of the medical sheet 300 to the recipient site 402. Subsequently, the transfer instrument 10 is withdrawn from the chest 408 with the first support portion 26 and the second support portion 50 retracted in the outer cylinder 22.

The first embodiment has the following effects.

It is possible to efficiently transfer, by moving the second shaft 48 along the first shaft 24 with the medical sheet 300 supported by the first support surface 261 and the second support surface 74 as illustrated in FIG. 12, the medical sheet 300 to the recipient site 402 (treatment site) (see FIG. 13).

As illustrated in FIG. 3, since the second support portion 50 has a shape tapered in the distal end direction, the contact area of the second support surface 74 with the medical sheet 300 (see FIG. 8) becomes smaller toward the distal end, and the frictional resistance between the medical sheet 300 and the second support surface 74 decreases accordingly. It is therefore possible to prevent, as illustrated in FIGS. 13 and 14, when the second shaft 48 is moved in the proximal end direction along the first shaft 24 after the medical sheet 300 is transferred to the recipient site 402, the medical sheet 300 from returning in the proximal end direction with the medical sheet 300 adhering to the second support surface 74. As a result, when the medical sheet 300 is placed in the recipient site 402 that is located in a narrow space in the living body, the medical sheet 300 can be transferred and placed in the recipient site 402 without using a support member such as forceps.

As illustrated in FIG. 3, since L2 is greater than or equal to 50% of L1 (L2≥L1/2), the contact area of the second support surface 74 with the medical sheet 300 at the tapered portion 100 becomes small. It is therefore possible to efficiently prevent, as illustrated in FIGS. 13 and 14, when the second shaft 48 is moved in the proximal end direction along the first shaft 24 after the medical sheet 300 is transferred to the recipient site 402, the medical sheet 300 from returning in the proximal end direction with the medical sheet 300 adhering to the second support surface 74.

As illustrated in FIG. 3, since the side edges 104 of both sides of the tapered portion 100 in the width direction (the direction of arrow W) linearly extend, and the distance L3 in the width direction between the side edges 104 of both sides decreases toward the distal end 102 of the second support portion 50, the tapered portion 100 can be formed in an acute triangular shape. It is therefore possible to easily reduce the contact area of the second support surface 74 with the medical sheet 300 (see FIG. 8) at the tapered portion 100.

Since the distal end edge 106 of the second support portion 50 has an arc shape, it is possible to prevent the distal end edge 106 of the second support portion 50 from damaging the recipient site 402 when the second shaft 48 is moved in the distal end direction along the first shaft 24. It is further possible to prevent breakage of the medical sheet 300 supported by the second support surface 74 when the medical sheet 300 comes into contact with the distal end edge 106 of the second support portion 50.

It is possible to efficiently transfer, by moving the second shaft 48 along the first shaft 24 with the medical sheet 300 supported on the second support surface 74 as illustrated in FIG. 12, the medical sheet 300 to the recipient site 402 (see FIG. 13).

As illustrated in FIG. 9, the first support portion 26 and the second support portion 50 can be retracted in the outer cylinder 22 in a compact form at the first position. Further, as illustrated in FIG. 3, since the second support portion 50 has a shape tapered in the distal end direction, it is possible to effectively prevent the second support portion 50 from becoming crinkled when the second support portion 50 is retracted in the outer cylinder 22.

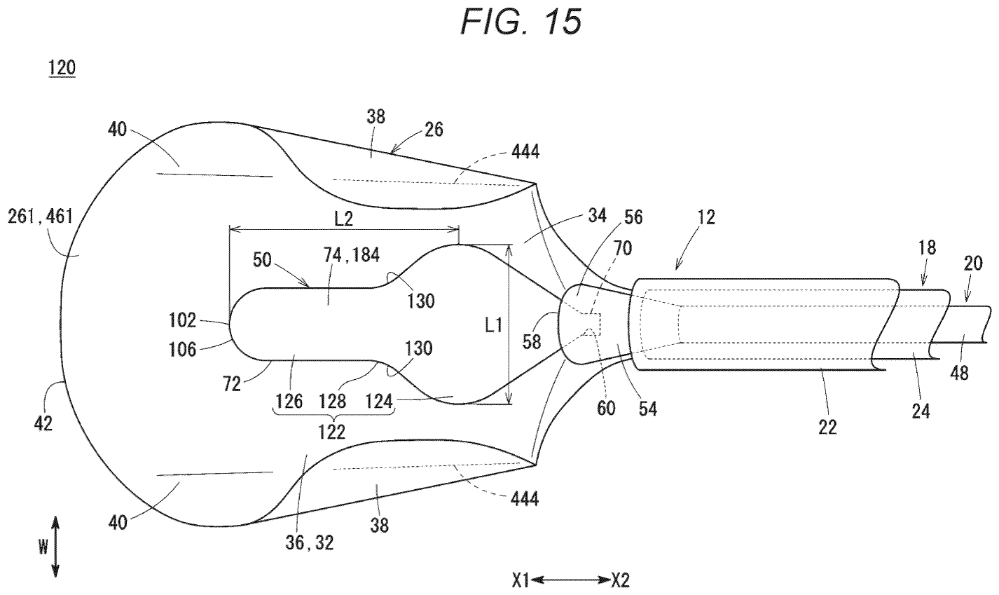

A transfer instrument 120 according to a second embodiment will be described with reference to FIG. 15. Components of the transfer instrument 120 according to the second embodiment that are the same as of the transfer instrument 10 (see FIGS. 1 to 14) according to the first embodiment are denoted by the same reference numerals, and a detailed description of such components will not be repeated.

FIG. 15 is a plan view of a distal end of the transfer instrument 120 according to the second embodiment. In the transfer instrument 120, the second support body 72 of the second support portion 50 has a tapered portion 122 tapered in the distal end direction. The tapered portion 122 includes a first portion 124, a second portion 126, and an intermediate portion 128. In the tapered portion 122, the first portion 124, the intermediate portion 128, and the second portion 126 are provided in this order in the distal end direction. The first portion 124 includes a portion with a maximum width L1 of the second support body 72 (tapered portion 122). The second portion 126 includes the distal end 102 and the distal end edge 106 of the second support portion 50 of the second support body 72. The second portion 126 protrudes from the intermediate portion 128 in the distal end direction. The intermediate portion 128 is a portion of the second support body 72 located between the first portion 124 and the second portion 126. Side edges 130 of both sides of the intermediate portion 128 in the width direction (the direction of arrow W) extend inward in the width direction. As extending in the distal end direction, each side edge 130 extends inward in the width direction. Therefore, the second portion 126 is shorter in length in the width direction than the first portion 124.

Effects of the second embodiment will be described. In the second embodiment, the tapered portion 122 includes the first portion 124, the intermediate portion 128, and the second portion 126, and the side edges 130 of both sides of the intermediate portion 128 extend inward in the width direction. This allows, in a simple manner, a reduction in the contact area of the second support surface 74 with the medical sheet 300 at the tapered portion 122.

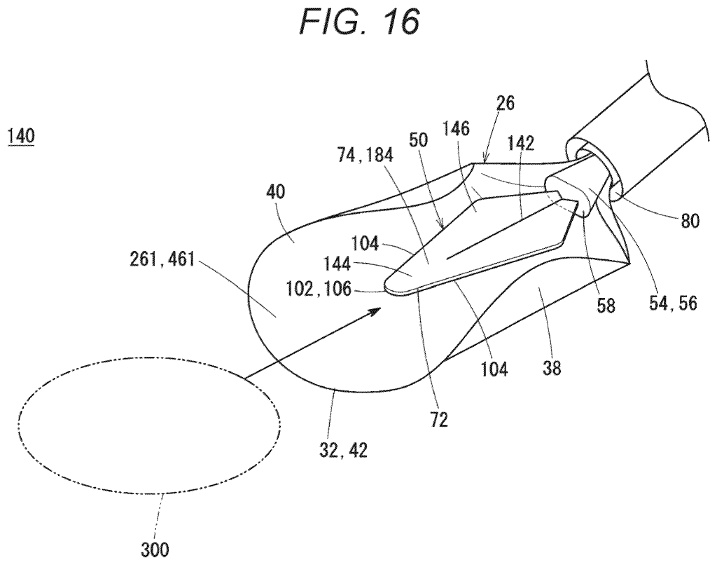

A transfer instrument 140 according to a third embodiment will be described with reference to FIG. 16.

FIG. 16 is a perspective view of a distal end of the transfer instrument 140 according to the third embodiment. In the transfer instrument 140, a center of the second support portion 50 in the width direction (the direction of arrow W illustrated in FIGS. 3 and 15) is bent or curved toward the first support portion 26. Specifically, in the second support portion 50, a center of the second support body 72 in the width direction is bent or curved toward the first support portion 26.

FIG. 16 illustrates a case where the center of the second support body 72 in the width direction is bent toward the first support portion 26. A fold line 142 is formed along the axial direction of the second shaft 48 at the center of the second support body 72 in the width direction. The fold line 142 is formed between the proximal end of the second support body 72 and a center of the second support body 72 in the axial direction of the second shaft 48. Accordingly, the center of the second support body 72 in the width direction is bent toward the first support portion 26.

Therefore, when both the first support portion 26 and the second support portion 50 are retracted in the outer cylinder 22 at the first position, the second support portion 50 is retracted in the outer cylinder 22 with the second support portion 50 curved and deformed into a round shape around the bent or curved center (see FIG. 10).

Further, a portion of the second support surface 74 between the portion bent or curved toward the first support portion 26 and the distal end 102 of the second support portion 50 includes a flat surface 144. In FIG. 16, the distal end of the second support surface 74 is the flat surface 144. In the third embodiment, the medical sheet 300 is supported on the flat surface 144 of the second support surface 74.

In the third embodiment, the second support body 72 of the second support portion 50 includes a tapered portion 146 tapered in the distal end direction.

Effects of the third embodiment will be described.

In the third embodiment, the center of the second support portion 50 in the width direction is bent or curved toward the first support portion 26. This allows the second support portion 50 to be easily curved and deformed at the first position into a round shape around the bent or curved center and retracted in the outer cylinder 22 (see FIG. 10). As a result, it is possible to effectively prevent the second support portion 50 from becoming crinkled when the second support portion 50 is retracted in the outer cylinder 22.

Further, since the portion of the second support surface 74 between the portion bent or curved toward the first support portion 26 and the distal end 102 of the second support portion 50 is the flat surface 144, the medical sheet 300 is easily supported by the flat surface 144.

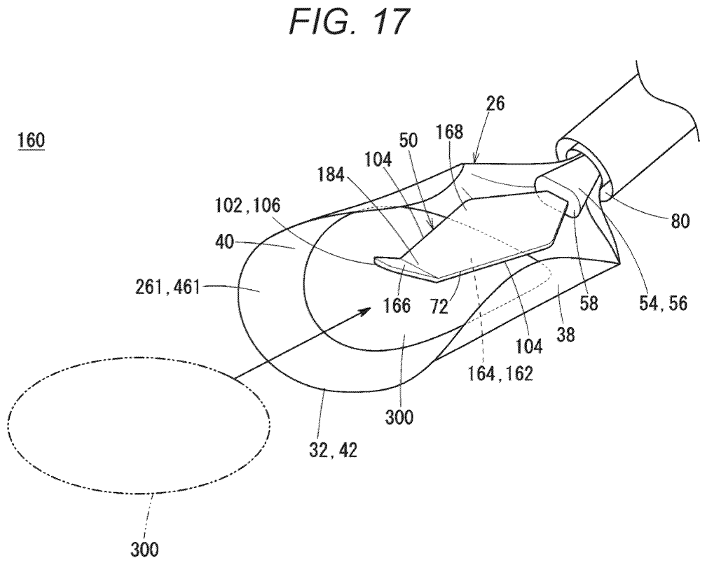

A transfer instrument 160 according to a fourth embodiment will be described with reference to FIGS. 17 to 19.

FIG. 17 is a perspective view of a distal end of the transfer instrument 160 according to the fourth embodiment. In the transfer instrument 160, a bottom surface 162 of the second support portion 50 facing the first support surface 261 serves as a second support surface 164. In the transfer instrument 160, the medical sheet 300 is supported between the first support surface 261 and the second support surface 164. That is, the medical sheet 300 is supported with the medical sheet 300 vertically sandwiched between the first support portion 26 and the second support portion 50. This causes the carrier holding portion 54 to press the medical sheet 300 in the distal end direction with a lower portion of the pressing surface 58 relative to the second support portion 50.

The second support portion 50 includes a bent portion 166. The bent portion 166 corresponds to the distal end of the second support portion 50. The bent portion 166 includes the distal end 102 of the second support portion 50. The bent portion 166 is formed by the distal end of the second support portion 50 bent in a direction (upward direction) away from the first support surface 261. It is desirable that the bent portion 166 be bent upward at any desired angle (for example, an angle in a range of 10° to 60°) relative to the second support body 72.

In the transfer instrument 160, the second support body 72 of the second support portion 50 includes a tapered portion 168. The tapered portion 168 has the same configuration as of the tapered portion 100 (see FIG. 1) except that the bent portion 166 is further provided.

Regarding a transfer method in the fourth embodiment, points different from the first embodiment (see FIGS. 1 to 14) will be described.

In the sheet placing process (step S2 in FIG. 6), the user lifts the bent portion 166 upward with tweezers (not illustrated) or the like. When the bent portion 166 is lifted upward, the second support body 72 is curved upward with the second joint 70 as a fulcrum. As a result, a gap is formed between the first support surface 261 and the second support surface 164. The user inserts the medical sheet 300 into the gap thus formed. After the medical sheet 300 is inserted into the gap, when the user returns the bent portion 166 to the original position, the medical sheet 300 is supported by the first support surface 261 and the second support surface 164.

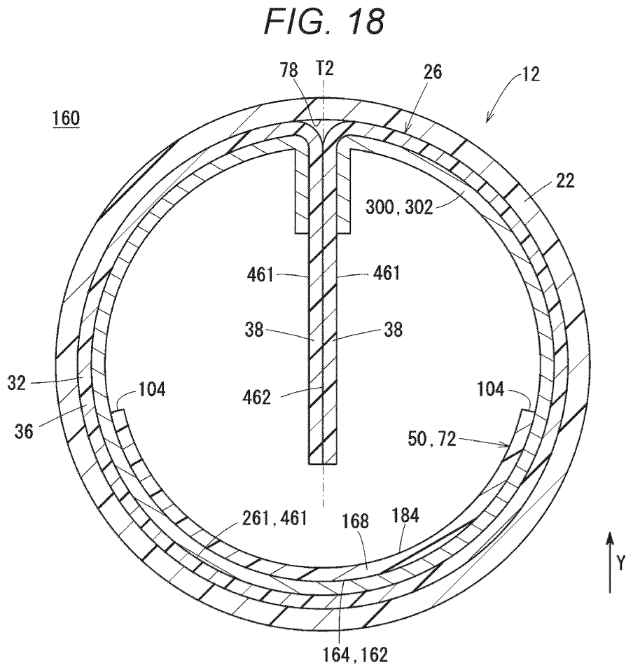

In the retracting process (step S3 in FIG. 6), as illustrated in FIG. 18, when both the first support portion 26 and the second support portion 50 are retracted in the outer cylinder 22 at the first position, the second support portion 50 is positioned below the free end of each first protrusion 38 in the outer cylinder 22. At this time, the first support portion 26 and the second support portion 50 are retracted in the outer cylinder 22 with the medical sheet 300 sandwiched between the first support portion 26 and the second support portion 50.

In the unfolding process (step S5 in FIG. 6), the medical sheet 300 is caused to protrude in the distal end direction from the distal-end opening 80 of the outer cylinder 22 with the medical sheet 300 supported by the first support surface 261 and the second support surface 164 at the second position.

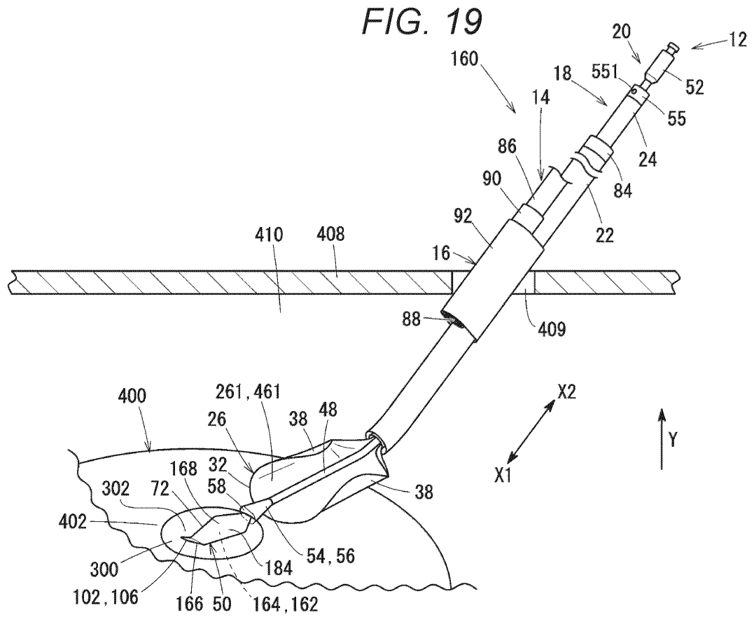

In the moving process (step S6 in FIG. 6), as illustrated in FIG. 19, when the second carrier member 20 is moved in the distal end direction (the direction of arrow X1) relative to the first carrier member 18, the carrier holding portion 54 presses the medical sheet 300 in the distal end direction with the lower portion of the pressing surface 58 relative to the second support portion 50 to position the whole of the medical sheet 300 further than the first support portion 26 in the distal end direction. As a result, the medical sheet 300 is moved to above the recipient site 402 of the heart 400.

Effects of the fourth embodiment will be described.

As illustrated in FIG. 17, since the medical sheet 300 is pressed in the distal end direction by the carrier holding portion 54 (pressing portion) when the second shaft 48 is moved along the first shaft 24 with the medical sheet 300 supported between the first support surface 261 and the second support surface 164, it is possible to efficiently transfer the medical sheet 300 to the recipient site 402 (treatment site).

Further, since the medical sheet 300 is supported in the vertical direction by the first support surface 261 and the second support surface 164, it is possible to transfer the medical sheet 300 to the recipient site 402 with the medical sheet 300 prevented from becoming crinkled. As a result, when the medical sheet 300 is placed in the recipient site 402 located in a narrow space in the living body, it is possible to transfer and place the medical sheet 300 to and in the recipient site 402 without using a support member such as forceps and with the medical sheet 300 prevented from becoming crinkled, breaking, and the like.

It is possible to easily cause, by lifting the bent portion 166 in a direction (upward direction) away from the first support surface 261, the second support portion 50 to curve in the direction (upward direction) away from the first support surface 261. Accordingly, a gap is formed between the first support surface 261 and the second support surface 164, so that it is possible to easily place the medical sheet 300 between the first support surface 261 and the second support surface 164.

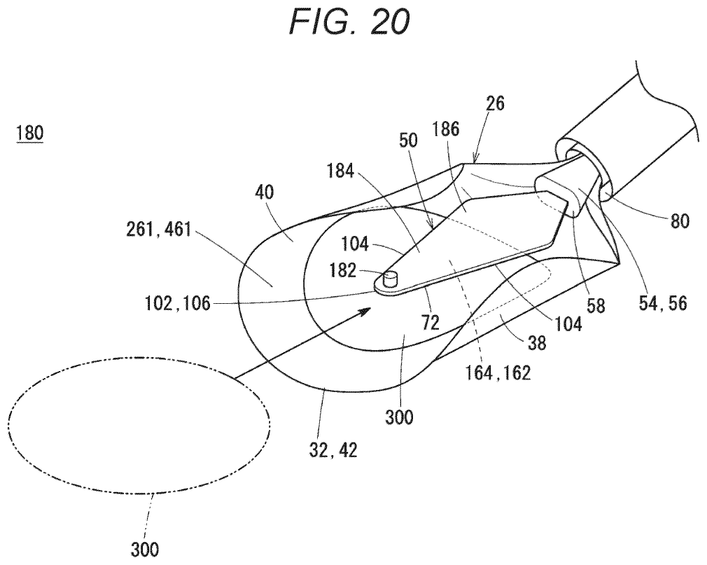

A transfer instrument 180 according to a fifth embodiment will be described with reference to FIG. 20.

FIG. 20 is a perspective view of a distal end of the transfer instrument 180 according to the fifth embodiment. The transfer instrument 180 according to the fifth embodiment is different from the transfer instrument 160 according to the fourth embodiment (see FIGS. 17 to 19) in that the second support portion 50 includes a projection 182 instead of the bent portion 166. The projection 182 is provided on the upper surface 184 of the second support body 72 of the second support portion 50. The projection 182 is provided at the distal end of the upper surface 184 of the second support body 72. The second support body 72 includes a tapered portion 186. The tapered portion 186 has the same configuration as of the tapered portion 100 (see FIG. 1).

In the fifth embodiment, when the user lifts the projection 182 upward with tweezers (not illustrated) or the like in the sheet placing process (step S2 in FIG. 6), the second support body 72 is curved upward with the second joint 70 as a fulcrum. As a result, a gap is formed between the first support surface 261 and the second support surface 164, so that the user can insert the medical sheet 300 into the gap thus formed. When the user returns the projection 182 to the original position after inserting the medical sheet 300 into the gap, the medical sheet 300 is supported by the first support surface 261 and the second support surface 164.

Effect of the fifth embodiment will be described. In the fifth embodiment, it is possible to easily cause, by lifting the projection 182 in a direction (upward direction) away from the first support surface 261, the second support portion 50 to curve in the direction (upward direction) away from the first support surface 261. Accordingly, a gap is formed between the first support surface 261 and the second support surface 164, so that it is possible to easily place the medical sheet 300 between the first support surface 261 and the second support surface 164.

The present invention is not limited to the above-described disclosure, and various configurations can be adopted without departing from the gist of the present invention. That is, the detailed description above describes embodiments of a transfer instrument and transfer method for transferring a medical sheet to a treatment site of a living body, representing examples of the new transfer instrument and transfer method disclosed here. But the invention is not limited to the precise embodiments and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

What is claimed is:

1. A transfer instrument used to transfer a medical sheet to a treatment site of a living body, the transfer instrument comprising:
a first carrier member including a first shaft and a first support portion, the first support portion being provided at a distal end of the first shaft and having a first support surface;
a second carrier member including a second shaft and a second support portion, the second shaft being positionable relative to the first shaft to extend along the first shaft and be movable along the first shaft, the second support portion being provided at a distal end of the second shaft and having a second support surface smaller than the first support surface;
the first support surface and the second support surface being configured to support the medical sheet; and
the second support portion having a shape tapered in a distal end direction.

2. The transfer instrument according to claim 1, wherein
the shape tapered in the distal end direction of the second support portion includes a tapered portion of the second support portion that is tapered in the distal end direction between a portion of the second support portion having a maximum width in a width direction orthogonal to an axial direction of the second shaft and a distal end of the second support portion, and
L2 is greater than or equal to 50% of L1, wherein L1 is the maximum width of the second support portion and L2 is a length of the tapered portion of the second support portion in the axial direction.

3. The transfer instrument according to claim 2, wherein
side edges of both sides of the tapered portion in the width direction linearly extend, and
a distance between the side edges of both sides in the width direction decreases toward the distal end of the second support portion.

4. The transfer instrument according to claim 2, wherein
the tapered portion includes a first portion including the portion of the second support portion having the maximum width, a second portion including the distal end of the second support portion, and an intermediate portion between the first portion and the second portion, and
side edges of both sides of the intermediate portion in the width direction extend inward in the width direction.

5. The transfer instrument according to claim 1, wherein a distal end edge of the second support portion has an arc shape.

6. The transfer instrument according to claim 1, wherein the second support surface is configured to support the medical sheet.

7. The transfer instrument according to claim 1, wherein
the second carrier member further includes a pressing portion provided at the distal end of the second shaft,
the second support portion is fixed to the pressing portion,
the first support surface includes an upper surface of the first support portion,
the second support surface includes a bottom surface of the second support portion, the bottom surface facing the first support surface,
the first support surface and the second support surface are configured so that the medical sheet is supportable between the first support surface and the second support surface, and
the pressing portion is configured to press, in the distal end direction, the medical sheet when the medical sheet is supported between the first support surface and the second support surface.

8. The transfer instrument according to claim 7, wherein the second support portion includes a bent portion that is bent in a direction away from the first support surface, the bent portion including a distal end of the second support portion.

9. The transfer instrument according to claim 7, wherein the second support portion includes a projection provided on an upper surface opposite to the second support surface.

10. The transfer instrument according to claim 1, further comprising an outer cylinder including a distal-end opening, wherein
the first shaft and the second shaft are positionable in the outer cylinder and are movable along the outer cylinder,
at a first position where the first shaft and the second shaft are moved in a proximal end direction relative to the outer cylinder to cause the first support portion and the second support portion to retract in the outer cylinder, the first support portion and the second support portion are retracted in the outer cylinder with the first support portion and the second support portion curved and deformed, and
at a second position where the first shaft and the second shaft are moved in the distal end direction relative to the outer cylinder to cause the first support portion and the second support portion to protrude from the distal-end opening, the first support portion and the second support portion move out from the outer cylinder in the distal end direction to unfold.

11. The transfer instrument according to claim 10, wherein
the second support portion is bent or curved toward the first support portion at a center of the second support portion in a width direction orthogonal to an axial direction of the second shaft, and
at the first position, the second support portion is retracted in the outer cylinder with the second support portion curved and deformed in a round shape around the center that is bent or curved.

12. The transfer instrument according to claim 11, wherein a portion of the second support surface between a portion bent or curved toward the first support portion and a distal end of the second support portion includes a flat surface.

13. A transfer instrument for transferring a medical sheet to a treatment site of a living body, the transfer instrument comprising:
an outer cylinder having a through hole that extends throughout the outer cylinder from a distal-end opening at a distal end of the outer cylinder to a proximal-end opening at a proximal end of the outer cylinder;
a first carrier member that includes a first shaft and a first support portion, the first shaft extending in an extending direction and having a distal end portion at which is fixed the first support portion so that the first shaft and the first support portion move together, the first support portion extending in a distal direction away from the first shaft and having a first support surface configured to support the medical sheet;
a second carrier member that includes a second shaft and a second support portion, the second shaft extending in the extending direction and having a distal end portion at which is fixed the second support portion so that the second shaft and the second support portion move together, the second support portion extending in a distal direction away from the second shaft and having a second support surface configured to support the medical sheet;
the first shaft and the second shaft being positionable in the through hole in the outer cylinder and being: i) axially movable in a proximal direction relative to the outer cylinder to move the first support portion and the second support portion from an extended position in which the first support portion and the second support portion are distal of the distal-end opening of the outer cylinder while supporting the medical sheet to a retracted position in which the first support portion and the second support portion are retracted into the through hole in the outer cylinder together with the medical sheet; and ii) axially movable in a distal direction relative to the outer cylinder to move the first support portion and the second support portion from the retracted position to the extended position;
the second support portion having a width orthogonal to the extending direction; and
the second support portion, in plan view when in the extended position, having a tapered portion that has a tapering shape in which the width of the tapered portion of the second support portion narrows in the distal direction.

14. The transfer instrument according to claim 13, wherein the first support portion has a width orthogonal to the extending direction, a maximum width of the first support portion being greater than a maximum width of the second support portion.

15. The transfer instrument according to claim 13, wherein the second support portion includes a bent portion that is bent in a direction away from the first support portion, the bent portion being a part of the tapered portion that has the tapering shape.

16. The transfer instrument according to claim 13, wherein the second support portion includes a surface facing away from the first support portion, and including an upstanding projection that projects away from the surface facing away from the first support portion.

17. The transfer instrument according to claim 13, wherein the second support portion includes a distal-most portion, a proximal-most portion and an intermediate portion between the distal-most portion and the proximal most portion, the intermediate portion being the tapered portion, width of the proximal-most portion of the second support portion increasing toward the intermediate portion.

18. The transfer instrument according to claim 13, wherein the second support portion is fixed to and extends distally away from a carrier holding portion, the carrier holding portion having a pressing surface that faces in the distal direction to contact a periphery of the medical sheet, the pressing surface projecting outwardly of a part of the second support portion immediately adjacent the pressing surface.

19. A method comprising:
moving a first support portion and a second support portion support portion toward a medical sheet, the first support portion being fixed to a distal end portion of a first shaft and extending in a distal direction away from the first shaft, the first support portion having a first support surface, the second support portion being fixed to a distal end portion of a second shaft that extends in an extending direction, the second support portion extending in a distal direction away from the second shaft, the second support portion being positioned in overlying relation to the first support portion, the second support portion having a width orthogonal to the extending direction, the second support portion having a tapered portion that has a tapering shape in which the width of the tapered portion of the second support portion narrows in the distal direction, the second support portion, inclusive of the tapered portion of the second support portion, having a second support surface; and
positioning the medical sheet so that the medical sheet is supported by the first support surface and the second support surface, inclusive of the second support surface of the tapered portion.

20. The method according to claim 19, wherein the positioning of the medical sheet includes positioning the medical sheet between ands in contact with the first support surface and the second support surface.
